(19) United States
(12) Patent Application Publication
(10) Pub. No.: US 2016/0015478 A1
(43) Pub. Date: Jan. 21, 2016

(54) SYSTEM AND METHOD FOR ACOUSTICAL ENDODONTICS

(71) Applicants: BRIGHAM AND WOMEN'S HOSPITAL, INC., Boston, MA (US); NEWPORT ENDODONTICS, P.C., Chestnut Hill, MA (US)

(72) Inventors: Ferenc A. Jolesz, Brookline, MA (US); Phillip Jason White, Boston, MA (US); Gregory Thomas Clement, Medford, MA (US); Nathan McDannold, Jamaica Plain, MA (US); Tom C. Pagonis, Chestnut Hill, MA (US); Douglass Adams, St. Augustine, FL (US)

(21) Appl. No.: 14/772,667

(22) PCT Filed: Mar. 3, 2014
(86) PCT No.: PCT/US14/19848

§ 371 Date: Sep. 3, 2015

Related U.S. Application Data
(60) Provisional application No. 61/773,905, filed on Mar. 7, 2013.

(51) Int. Cl. A61C 1/07; A61B 8/08; A61C 1/00; A61C 5/02

(52) U.S. Cl. CPC A61C 1/07; A61C 5/02; A61B 8/0875; A61C 1/0015; A61N 2007/0026

(57) ABSTRACT

System and method for non-invasive sonication, through at least one of enamel and dentin, of a root-canal system of a target tooth with an externally delivered ultrasound beam, to disinfect the root-canal system while blocking a nerve associated with the tooth and removing a need for radiography to confirm the results of endodontics. The tooth does not have a physical access opening to the root canal system. When focused, the beam is oriented to expose the root-canal system to the ultrasound within the confocal range of the beam such as to concentrate the ultrasound energy within the root-canal system and to avoid irradiating teeth that are immediately adjacent to the target tooth.

### CROSS-REFERENCE TO RELATED APPLICATIONS

The present application claims priority from and benefit of the U.S. Provisional Patent Application No. 61/773,905 filed on Mar. 07, 2013 and titled “System and Method for Acoustical Endodontics”. The disclosure of the above-identified provisional patent application is incorporated herein by reference in its entirety.

### TECHNICAL FIELD

The present invention relates to the use of ultrasound and, more particularly, to the application of ultrasound to an intact tooth to effect microbial destruction and/or disinfection) thereof.

### BACKGROUND ART

Endodontics is the branch of dentistry which is concerned with the morphology, physiology and pathology of the human dental pulp and periradicular tissues. Its study and practice encompass the basic and clinical sciences including biology of the normal pulp, the etiology, diagnosis, prevention and treatment of diseases and injuries of the pulp and associated periradicular conditions. (American Dental Association, Adopted December 1983). Endodontic procedure(s) is a sequence of treatment for the pulp of a tooth which results in the elimination of microbial infection. This set of procedures is commonly referred to as a “root canal”, and is routinely performed over 41,000 times a day in the U.S. (as reported by The American Association of Endodontists). The established method for performing root canals comprises a sequence of well-established steps: (1) accessing pulpal tissue by drilling through enamel and dentin with engine-driven high speed turbine dental handpieces; (2) chemomechanical debridement of pulpal remnants and bacteria using hand or engine-driven rotary endodontic instrumentation (for example, files); and (3) the use of a variety of antimicrobial chemical irrigants, including sodium hypochlorite (NaOCl), ethylenediaminetetraacetic acid, or chlorhexidine. In addition, for cases that involve viable and active nerves, the typical standard-of-care includes pre-procedural application (usually by local anesthesia injection) and intra-procedural maintenance of anesthesia. In the cases of high levels of infection, (such as pulpal diagnoses of symptomatic necrosis with acute periradicular abscess, for example) treatment over multiple appointments is required with the administration of intracanal medicaments as inter-appointment dressings. Adjunctive treatment protocols include the use of sonic and ultrasonic activation and distribution of chemical irrigants.

Barring the incorporation of incremental technological and biomaterial/chemical advances on visualization (for example, dental microscope), instrumentation (such as engine-driven NiTi rotary files), irrigation (proprietary mixtures of chemical irrigants), and obturation (introduction of obturation materials and techniques), this sequence of chemo-mechanical treatments has largely remained invasive and unchanged for several decades, statistically demonstrating the success rate between about 65% and about 85% (see Ng, Y. et al., in International Endodontic Journal 40, 921-939, 2007). Therefore, there exists a need in endodontic system and method of devised to mitigate a number of invasive actions, confounding factors, and complications associated with the standard methods for anesthesia and the established methods for chemomechanical therapy.

### SUMMARY

Embodiments of the present invention provide a method for operating a source of ultrasound. The method comprises a step of transmitting a focused ultrasound beam generated by the source through a first tooth that is devoid of an access opening to an internal target area, which includes a root-canal system of the first tooth, and wherein the source defines a confocal range of the beam that overlaps with the internal target area. The method further comprises a step of maintaining such transmission of the focused ultrasound beam through the first tooth for a period of time sufficient to disinfect the root canal system of the first tooth. The step of maintaining may include keeping the focused acoustic beam transmitted through the first tooth for a period of time sufficient to treat at least one of (i) peri-implantitis in implant dentistry and (ii) ADA/AAP Case types I-V of periodontal disease. In a specific case, the method of the invention is used for treatment, of at least one of peri-implantitis in implant dentistry and ADA/AAP Case types I-V of periodontal disease.

Embodiments of the invention additionally provide a method for determining a parameter characterizing a tooth tissue. The method includes (i) sonicating a first tooth with a focused ultrasound beam incident on the first tooth externally such that an internal target area of the tooth is within a confocal range of the of the focused beam, where the internal target area includes a root-canal system of the tooth; and (ii) receiving, with a detector unit, a portion of the focused beam that has irradiated the first tooth to collect ultrasound data representing interaction of the focused beam with the tooth. The method further includes determining, from the collected data, at least one of sound speed, mass density, and attenuation values associated with the tooth tissue.

Embodiments of the invention additionally provide an article of manufacture that includes a source of ultrasound including means defining, in operation, a focused ultrasound beam generated by the source; and a non-transitory tangible computer-readable medium having computer-readable program code thereon. The computer-readable program code contains program code for acquiring data that represent the focused ultrasound beam that has been transmitted through a tooth devoid of access opening to a root-canal system of the tooth; and program code for determining, from the acquired data, a parameter characterizing a process of sonication of the tooth with the focused ultrasound beam in such a manner as to disinfect the root-canal system.

### BRIEF DESCRIPTION OF THE DRAWINGS

The invention will be more fully understood by referring to the following Detailed Description in conjunction with the generally not to scale Drawings, of which:

FIG. 1 is a diagram schematically illustrating sonication of an intact tooth with focused ultrasound beam according to an embodiment of the invention;

FIG. 2 is a series of video frames illustrating transformation of material contained in a root-canal area by application of the focused ultrasound beam according to an embodiment of the invention;

FIG. 3 is a plot illustrating substantial spatial confinement of a focal region of a focused ultrasound beam within the root-canal area of the tooth (an image overlay of ex vivo human molar CT with intensity field showing ultrasound energy focus in the root canal);

FIG. 4 illustrates an embodiment of the apparatus for HIFU treatment with supporting peripheral equipment;

FIG. 5 is a diagram illustrating the use of the embodiment of FIG. 4 during endodontic treatment as disclosed herein;

FIG. 6A illustrates a variety of interchangeable tips for HIFU device structured to treat both peri-implantitis and a periodontal disease;

FIG. 6B is a view of the HIFU device equipped with one of the interchangeable tips of FIG. 6A for accessing a periodontal pocket for HIFU disinfection;

FIG. 7 is a flow-chart illustrating an embodiment of the method for application of a source of acoustic beam according to the invention.

### DETAILED DESCRIPTION

References throughout this specification to “one embodiment,” “an embodiment,” “a related embodiment,” or similar language mean that a particular feature, structure, or characteristic described in connection with the referred to “embodiment” is included in at least one embodiment of the present invention. Thus, appearances of the phrases “in one embodiment,” “in an embodiment,” and similar language throughout this specification may, but do not necessarily, all refer to the same embodiment. It is to be understood that no portion of disclosure, taken on its own and in possible connection with a figure, is intended to provide a complete description of all features of the invention.

In addition, the following disclosure may describe features of the invention with reference to corresponding drawings, in which like numbers represent the same or similar elements wherever possible. In the drawings, the depicted structural elements are generally not to scale, and certain components are enlarged relative to the other components for purposes of emphasis and understanding. It is to be understood that no single drawing is intended to support a complete description of all features of the invention. In other words, a given drawing is generally descriptive of only some, and generally not all, features of the invention. A given drawing and an associated portion of the disclosure containing a description referencing such drawing do not, generally, contain all elements of a particular view or all features that can be presented is this view, for purposes of simplifying the given drawing and discussion, and to direct the discussion to particular elements that are featured in this drawing. A skilled artisan will recognize that the invention may possibly be practiced without one or more of the specific features, elements, components, structures, details, or characteristics, or with the use of other methods, components, materials, and so forth. Therefore, although a particular detail of an embodiment of the invention may not be necessarily shown in each and every drawing describing such embodiment, the presence of this detail in the drawing may be implied unless the context of the description requires otherwise. In other instances, well known structures, details, materials, or operations may be not shown in a given drawing or described in detail to avoid obscuring aspects of an embodiment of the invention that are being discussed.

Furthermore, the described features, structures, or characteristics of the invention may be combined in any suitable manner in one or more embodiments. Moreover, if the schematic flowchart diagram is included, it is generally set forth as a logical flowchart diagram. As such, the depicted order and labeled steps of the logical flow are indicative of one embodiment of the presented method. Other steps and methods may be conceived that are equivalent in function, logic, or effect to one or more steps, or portions thereof, of the illustrated method. Additionally, the format and symbols employed are provided to explain the logical steps of the method and are understood not to limit the scope of the method. Although various arrow types and line types may be employed in the flowchart diagrams, they are understood not to limit the scope of the corresponding method. Indeed, some arrows or other connectors may be used to indicate only the logical flow of the method. For instance, an arrow may indicate a waiting or monitoring period of unspecified duration between enumerated steps of the depicted method. Without loss of generality, the order in which processing steps or particular methods occur may or may not strictly adhere to the order of the corresponding steps shown. The invention as recited in claims appended to this disclosure is intended to be assessed in light of the disclosure as a whole.

In accordance with embodiments of the present invention, method and apparatus are disclosed configured to deliver acoustic energy towards and through an intact tooth to effectuate mechanical fractionation (for example, as mediated by induced acoustic cavitation) and or thermal effect (for example, through nonlinear adiabatic heating) to effectuate reduction in microbial population in a root canal area, provide non-invasive anesthesia of the target area, and, optionally, non-invasive denervation of the tooth. The application of the proposed system and method fundamentally changes conventionally invasive endodontics to non-invasive. It obviates the need in having a physical portal (such as a drilled dental access opening).

In particular, the use of high-intensity focused ultrasound (HIFU) in accordance with embodiments of the invention mitigates a number of invasive actions, confounding factors, and complications associated with the standard methods for anesthesia and the established methods for chemomechanical therapy, such as, for example, those using invasive methods such as nerve blocking, access opening, mechanical cleaning and shaping of the tooth, exposure of the treated tooth to chemical irrigants, adjunctive intracanal sonic or ultrasonic activation, obturation (filling) of the root canal system with biomaterials and placement of a final restoration (i.e., crown).

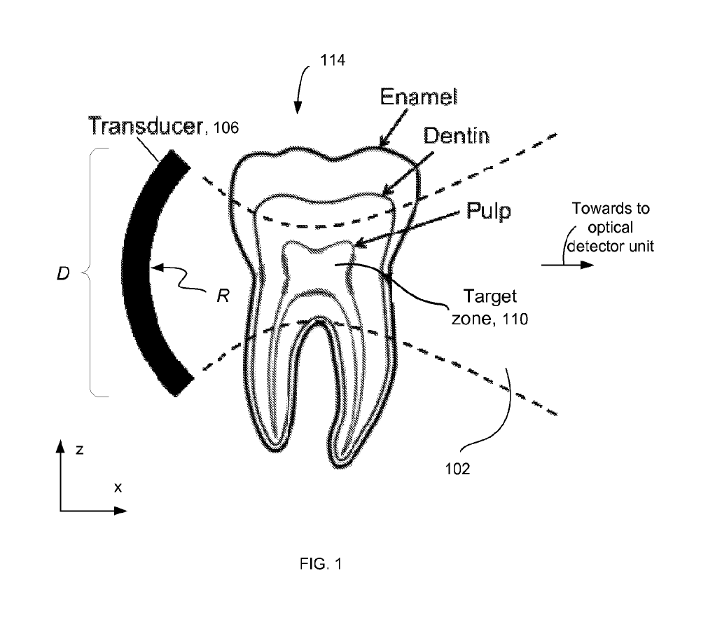

FIG. 1 is a diagram illustrating a schematic example of the proposed application of focused beam 102 of ultrasound, generated by a geometrically-curved transducer 106, through intact enamel and dentin to an internal target zone 110 of an intact tooth 114. For the focused ultrasound beam 102, the sonication center frequency and the transducer's f-number

$$F = \frac{{R}_{c}}{D}$$

(where F is the focal number, R_{c} is the radius of curvature of the transducer 106, and D is the diameter of the aperture of the transducer 106) largely determine the spatial distribution of the ultrasound irradiance. A skilled artisan will readily appreciate that in a related embodiment (not shown) an array of ultrasound transducers, for example a phased array, can be used instead of a single spatially curved transducer 106 to form a focused ultrasound beam 102. In yet another related embodiment (not shown), a source of ultrasound can be used that enables the generation of a substantially non-focused ultrasound beam that is later, upon propagation, focused with the use of focusing means such as elements and systems that refract and/or reflect ultrasound (for example, ultrasound lenses and reflectors) while changing a curvature of the ultrasound wavefront propagating from the source. In one implementation, the transducer(s) can be dimensioned to achieve localization of about 90% of the energy of the acoustic beam 102 within the internal target area that is substantially defined by average spatial extent of root canal system for each category of human teeth (viz., incisor, canine, premolar, and molar teeth).

The high sound speed, mass density, and attenuation values associated with anatomical solid elastic structures such as bone, enamel, and dentin are typically considered impediments for ultrasound applications, especially in ultrasound imaging, where relatively high-frequency (for example, between about 2 MHz and about 10 MHz) ultrasound signals must pass through the tooth structure to form a meaningful image. For the purposes of focused insonation of an intact tooth, however, and in stark contradistinction with typical ultrasound imaging parameters, the frequency (of sonication) is lower by about an order of magnitude and can be as low as hundreds of kHz, thereby allowing for the operational procedure.

In further reference to FIG. 1, a detector unit (not shown) may be disposed to receive at least a portion of the ultrasound beam 102 that has interacted with the tooth 114 and to collect data representing the speed, and/or impedance, and/or attenuation of the ultrasound in the tooth tissue as well as geometry-based effects on ultrasound transmission through the tooth to define combination of tooth-sonication parameters that is optimal for (1) maximal level of disinfection within the root canal, and (2) minimal damage to neighboring healthy tissues. To verify the optimal sonication procedure, micro-CT (Computed Tomography) scans of the irradiated tooth can be performed to determine internal variation in mass density as a result of ultrasound sonication for comparison with other experimental and simulation data.

Referring again to FIG. 1, optionally, the transducer(s) 106 are air-backed (for narrow-band high-frequency throughput operation), and the free-field (degassed water at standard temperature and pressure, STP, which is about 20 degrees ° C. and about 101.325 kPa) radiation profile of the completed transducers is measured with a pressure-sensitive needle hydrophone scanning unit (not shown). Upon confirmation of the measured pressure field with simulated predictions, similar measurements are performed with a series of ex vivo human dental specimens positioned within the Raleigh range of the ultrasound beam. The term “Rayleigh range” used herein describes a longitudinal (axial) extent of a focal region (focal range) of the ultrasound beam and denotes the distance along the propagation direction of an ultrasound beam from its waist (which substantially corresponds to the narrowest cross-section of the beam) to the place where the area of the cross section of the beam is approximately doubled. A related parameter is the confocal range or parameter, which is used to refer to twice the Rayleigh range.

These measurements can then be repeated for different spatial orientations of the tooth 114 (for example, to effectuate insonation through the lateral aspects of the tooth as well as sonication through the occlusal/incisal aspects of the tooth) over a statistically significant number of specimens (in one example, at least ten). Post-transmission and after removal of the tooth from the beam, the tooth-irradiating ultrasound beam is scanned to determine the beam profile at the focal region (for example, with the use of a k-space back projection method described by Clement et al. in J. Acoust. Soc. Am. 108, 441-446, 2000) for comparison with simulation data.

The simulation of ultrasound transmission through the layers of dental tissues can be performed using, for example, a modified wavevector space propagation method to provide data assisting selection of the optimal transducer geometries and frequencies and a method of verification of the experimental design.

A person of ordinary skill in the art will appreciate that the proposed method for use of ultrasound for endodontic treatment fundamentally deviates (both in the energy delivery pathway and the basic antimicrobial mechanism) from traditional applications of ultrasound during endodontic procedures. The reported up-to-date endodontic applications of ultrasound energy require the insertion of a probe (or a wire or a file) through a physical opening in the tooth's enamel and dentin and delivery of the ultrasound energy via the elastic structure of so-inserted probe to produce nonlinear fluid streaming within the medium in which it resides (as discussed, for example, by Van der Sluis, L. W. M., et al., in Passive ultrasonic irrigation of the root canal: a review of the literature. International Endodontic Journal 40, 415-426, 2007). This type of ultrasound-mediated irrigation acts, together with a chemical irrigant (typically NaOCl), to remove (i.e., not destroy) microorganisms from the inner walls of the root canal. The applicability of this invasive technique is limited at least in part by the inaccessibility of root canal geometries. In contradistinction with the accepted approaches, the proposed system and method deliver ultrasound energy non-invasively, in a focused fashion to the targeted location within the root canal. Cavitation (which involves the creation of vapor or gas cavities such as bubbles within tissue in response to the large negative pressures of an acoustic wave) can occur naturally only in a fluid or fluid-like medium, so by this fact will be an effect physically isolated within the root canals and its branches. Once induced, the cavities (or a collection of cavities) will either maintain an oscillatory pattern that linearly tracks the changing acoustic pressure field (i.e., non-inertial cavitation), or behave nonlinearly as the driving pressure oscillations increase in amplitude (i.e., inertial cavitation). Inertial cavitation involves the rapid implosion of the nucleated cavities, and as such, produces more intense mechanical action (i.e., shock waves and high-velocity jets) for tissue destruction. The controlled inception of non-inertial cavitation within diseased root canal systems is expected to provide the necessary mechanism for microbial tissue destruction and disinfection. The successful demonstration of an optimized delivery of antimicrobial energy to the target without damaging healthy periradicular tissues would represent a significant and innovative breakthrough for endodontic therapy and disruptive innovation in endodontics.

Exposure of a target area of a tooth to focused ultrasound beam according to an embodiment of the invention facilitates non-invasive disinfection of the root-canal system without removal of any matter therefrom, does not require a complementary imaging of the tooth that is used to confirm the results of conventionally performed endodontics, performs disinfection not only of a main root-canal channel but any mechanically or chemically inaccessible areas of the root canal system (i.e, lateral canals, fins, ramifications) that is exposed to the ultrasound beam contemporaneously with the main root-canal channel, and reduces the cost of the procedure by blocking the nerve conduction without a need in application of anesthetic and by completing treatment in substantially less time (for example, minutes instead of an hour).

Non-Invasive Disinfection. Determination of active disinfection of the targeted area within the root canal system of a tooth can be tested, for example, with the use of cultured mono-species endodontic pathogens (E. faecalis) in planktonic phase, multi-species endodontic pathogens in planktonic phase, and both mono- and multi-species endodontic pathogens as biofilms with intentional (experimental) infection of ex vivo human teeth and with naturally infected ex vivo human teeth sonicated with focused ultrasound according to an embodiment of the invention

As one example, the destruction of the population of planktonic phase and microbial biofilms can be measured, in 1 ml-cuvettes, with a spectrophotometer (at wavelength of about 600 nm, the 0.1 optical density unit corresponds to approximately 108 cells/ml). The preparation of bacterial cultures can be performed as follows: Aliquots of bacterial suspensions (108/ml) are placed in sterile microcentrifuge tubes and centrifuged (7000 rpm for 4 minutes). The supernatants are aspirated to a final volume of about 1 ml. Cultures are then placed in the wells of 24-well plates and subjected to focused ultrasound. After ultrasound sonication of the appropriate wells, serial dilutions of the contents of each well are prepared in BHI broth, and 100-ml aliquots are spread over the surfaces of blood agar plates. Survival fractions in each well are calculated by counting the colonies on the plates and dividing by the number of colonies from controls that are kept at room temperature for a period equal to the ultrasound time. The average survival fractions of the three wells per ultrasound group are determined in each experiment, and summary data are obtained by calculating the mean standard error average from 2 to 4 experiments for each microorganism. Differences between means are analyzed for statistical significance (by Student's t-test, for example).

To assess the antimicrobial effects of ultrasound sonication carried out according to an embodiment of the invention, within the root canal, an infected (maxillary and mandibular) ex vivo tooth that has been insonated can be studied to analyze microbial culture. For example, after sonication, the canals of each tooth can be completely filled with pre-reduced anaerobically sterilized (PRAS) Ringer's solution by using a sterile ProRinse 30-gauge irrigation needle; a sample can be collected by introducing an ISO size 10 K-type file to a working length of 0.5 mm short of the apical foramen and then agitated in the canal solution in the canal for 60 seconds. The file is then removed, and the file handle will be cut off under aseptic conditions and put in a 1.5-mL microcentrifuge tube containing 1 mL PRAS Ringer's solution.

Parameters of sonication of a tooth can optionally be iteratively adjusted with each series of experiments to determine ultrasound irradiance thresholds for producing a sufficient level of disinfection for a given type of microbe cells. “Sufficiency” of disinfection is defined as the level of bacteria removal and/or killing that is comparable to that achieved with the established chemomechanical technique for endodontic therapy.

Nerve Blocking. While the ability for ultrasound to block peripheral nerve conduction in general has been recognized, and while thermal and mechanical underlying mechanisms have been suggested to cause this effect, there does not appear to be general consensus on the cause of nerve blocking. Ultrasound has not become a standard method for providing local anesthesia due to the potential of inducing permanent nerve damage (as evidenced, for example, by Foley, J. L et al, in Image-guided high-intensity focused ultrasound for conduction block of peripheral nerves, Annals of biomedical engineering 35, 109-119, 2007; Colucci, V. et al., in Focused ultrasound effects on nerve action potential in vitro, Ultrasound Med. Biol. 35, 1737-1747, 2009). At the same time, to the best knowledge of the inventors, there is no existing precedent that has indicated the necessity of maintaining nerve tissue viability within the root canals after endodontic therapy. The endodontic application proposed in this disclosure falls within the category for which permanent alteration of nerve conduction is substantially inconsequential. In the standard procedure, nerve fibers within the root canals are removed along will all pulpal tissues, so there is no remnant nervous tissue. This scenario is functionally equivalent to a permanent damage of intracanal nerves. Accordingly, ultrasound-actuated nerve blocking or anesthesia (even if potentially permanent) is a suitable mechanism for replacing the traditional injection method of local anesthesia. According to an embodiment of the method of the invention, ultrasound sonication parameters that may potentially allow for providing temporary local anesthesia (applicable in other forms of dental application) are devised.

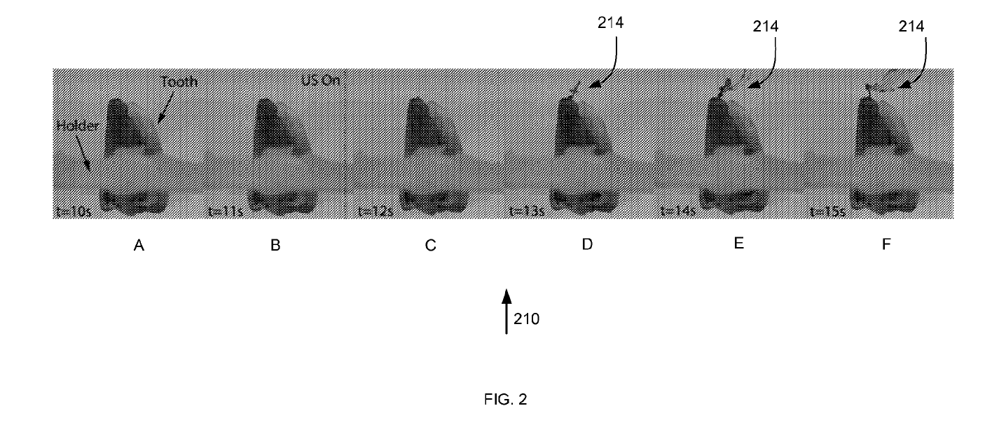

To this end, FIG. 2 illustrates a series of frames (A . . . E) presenting images of an intact ex vivo human tooth (molar) the root canal of which is filled with blue dye. The tooth is irradiated (sonicated) with high-intensity focused ultrasound (at a central frequency of f_{c}=0.272 MHz). Ultrasound penetrated the tooth from below, along the direction indicated with an arrow 210, to create a nonlinear jetting 214 of dye from the apical foramen (as shown in frames D, E, and F). This demonstrates that focused ultrasound beam non-invasively penetrates into the root canal to generate a measurable effect of transforming matter inside the tooth. Given the high ultrasound-absorption indices (on the order of 22 dB/cm at 1 MHz, for example) of dental enamel and dentin and the likelihood that higher levels of energy will be required to elicit a permanent nerve block, in devising the parameters of insonation appropriate for inducing nerve blocking the temperature at several points over the surface of the dental enamel should be monitored (for example, with small gauge thermocouples throughout the experiments). Micro-CT scans of pre- and post-sonication specimens will be obtained to verify the induction of cavitation within root canals and the subsequent destruction of tissue.

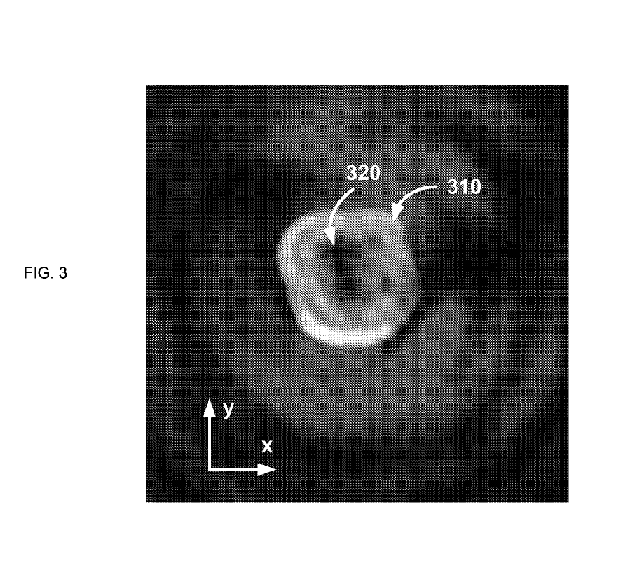

Energy Isolation. It is appreciated that a certain degree of ultrasound coupling between neighboring or adjacent teeth is unavoidable during the in vivo insonation of a particular tooth. Pathways for vibrational energy coupling from one root canal system to another may include tooth-to-tooth transmission via physical contact between the teeth (direct coupling) and via the elastic structure of the bone (i.e., either the mandible or maxilla) in which the teeth reside (indirect coupling). According to an embodiment of the invention, ultrasound sonication of a tooth is configured to ensure that delivery of irradiating energy to adjacent teeth in the oral cavity and/or to the healthy tissue is minimized such that the irradiated tooth remains substantially isolated within the intended target. The contour plot of FIG. 3 shows the overlay of a CT-image of ex vivo human molar 310 (in grayscale) with spatial distribution of ultrasound irradiance, in back-projection measurement (in pseudo color), demonstrating spatial isolation of the focal region 320 of the ultrasound beam within the root canal of the tooth 310.

In one implementation of the method of the invention, the sought after parameters of insonation are determined by (i) substantially concurrent with sonication scanning of ex vivo human teeth that are mounted in their anatomical locations within the mandible or maxilla; and (ii) performing k-space back-projected reconstruction on sets of data acquired with such substantially concurrent with sonication scanning to determine spatial distributions of ultrasound sonication within the target. Experimental data may be collected for multiple teeth mounted in at least three (3) mandibles and at least three (3) maxillae. In addition, optionally, experimental tests may be performed with both soft-tissue phantoms and store-acquired tissues to ascertain energy localization in situ (i.e., with ex vivo human teeth-and-mandible and teeth-and-maxilla specimens). In one experiment, the root canal systems for all teeth are filled with tissue phantoms that approximate the tissue absorption properties of pulpal tissue. High-intensity ultrasound is then directed into a targeted root canal system to induce cavitation and fractionation of the phantom material. The results are examined both by macroscopic and microscopic visualization and by micro-CT scanning.

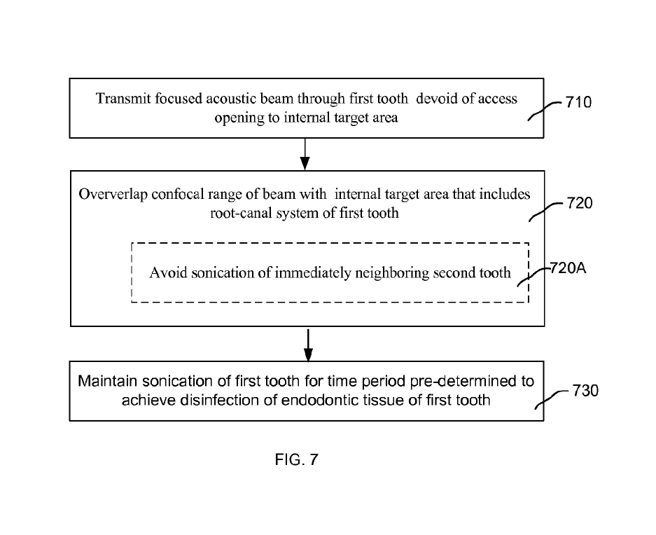

FIG. 7 provides a schematic flow-chart illustrating an embodiment of a method for operating a source of acoustic energy to achieve the goals discussed herein, according to an embodiment of the invention. At step 710, a focused acoustic beam generated by the source is transmitted through a target tooth (that is devoid of an access opening) to an internal target area. At step 720, a confocal range of so transmitted beam is overlapped with the internal target area that may include a root-canal of the target tooth. In a specific case, such spatial overlap is achieved at step 720A without sonication of immediately neighboring second tooth. Additionally, the method includes maintaining transmission of the focused beam through the target tooth, at step 730, for a period of time determined to be sufficient to achieve the goal of sonication.

It is appreciated, therefore that embodiments of the invention mitigate a number of invasive actions, confounding factors, and complications associated with the standard methods for anesthesia and the established methods for chemomechanical therapy. In particular:

With respect to Nerve Blocking (producing local dental anesthesia): The replacement of injection-based anesthesia with a noninvasive ultrasound-based method to block nerve conduction reduces and substantially eliminates the risk of complications associated with pre- and intra-procedural anesthesia (which include local paresthesias, trismus, hematomas, pain on injection, needle breakage, soft tissue injury, facial nerve paralysis, infection, and mucosal lesions) and alleviates prominent patient anxiety associated with dental visits and that has been shown to be largely correlated with fear of injection.

With respect to use of Radiography: Currently, radiography is used during endodontic treatment to determine and verify the correct length of canal files and subsequent obturation (filling of the canal). In the implementation of the present invention, which results in not having to put files into a tooth, the need for approximately two (2) to three (3) periapical radiographs per procedure is eliminated. While at an average effective dose of 5 μSv (0.5 mrem) per intraoral radiograph, this reduction in radiation exposure would not necessarily have a large impact per procedure, but, given the number of procedures that are performed per individual over a lifetime, and the number of procedures performed over an entire population, this reduction in radiation exposure could have a measurable epidemiological impact. More immediately, the elimination of intra- and post-surgical radiography would dramatically simplify the overall procedure to which a patient is subjected. Postsurgical radiographs are now taken to confirm that the root canal obturation is properly placed. Since the application of focused ultrasound according to the invention substantially eliminates the need for tooth access, chemo-mechanical debridement, and subsequent filling, the postsurgical radiograph becomes unnecessary.

With respect to Access Opening: The application of the invention removes the need for invasively creating a physical access opening in a tooth (for example, with high-speed drilling), through which the ambient and the root-canal system are in fluid communication. In other words, the subject tooth is devoid of an access opening through which a gas or fluid can penetrate between the ambient and a root canal system. The elimination of this requirement preserves the structural integrity of post-procedural teeth and, therefore, obviates the need in following restorative work (such as root canal obturation and crown placement, for example). Perhaps equally notable is the patient anxiety that is associated with the dental drill. The anxiety-producing effect of the dental drill has been well studied and for many patients often supersedes the anxiety of intraoral injection

With respect to Mechanical Cleaning and Shaping: Mechanical removal of diseased pulpal tissue and enlargement of the root canal system for subsequent obturation of the root canal space is substantially eliminated and, therefore, the complications of separating or breaking a file within the canal are mitigated as well. The use of embodiments of the invention additionally mitigates potential complications associated with iatrogenic misshaping of the root canals (e.g., “ledging”, “zipping”, “stripping”, or “transporting the apex”), which leads to variable outcomes because the canals become more difficult to fill. It also mitigates a complication of perforation (i.e., the file is pushed out the side of the canal).

With respect to Complex Anatomy of the Root-Canal System: The complex anatomy of the root canal system with its isthmuses, fins, ramifications, and lateral canals (microchannels) make complete debridement almost impossible, even when current methods of instrumentation and irrigation are performed to the highest technical standards. The application of focused ultrasound sonication to an intact tooth provides disinfection of minute branches of this complex anatomy, which is currently inaccessible with conventional endodontic chemomechanical methods.

With respect to Irrigation of the Tooth (the use of a variety of chemical irrigants such as sodium hypochlorite (NaOCl), ethylenediaminetetraacetic acid, or chlorhexidine, to disinfect and remove pulpal remnants). Complications associated with irrigation include the inadvertent delivery of NaOCl beyond the root structure into the mandible or maxilla, causing a severe and painful reaction. The ultrasound-based procedure of the invention does not require irrigation, and therefore, eliminates the possibility of this complication.

Example of Implementation of the system of the invention. The system is structured to allow a user to deliver ultrasound energy in a controlled fashion into the root canals and surrounding tissues of a patient's tooth. The delivered energy provides, either at the same time or at separate times, (i) anesthesia to the local area, and (ii) disinfection and/or sterilization of the targeted root canal system. To achieve these goals, the system comprises two distinct structural elements (1) a probe that is either handheld or mounted on an adjustable gantry, and (2) an electronic driving system (EDS) operably communicated with such probe. The following identifies but three possible structural configurations of the system: (a) the probe and EDS are coupled via electric wire such that the probe is operated by the user while the EDS remains at a distance (e.g., on the floor or on a benchtop away from the patient); (b) the probe is physically integrated with the EDS such that only direct wiring from line power is required for operation; and (3) the probe, EDS, and an on-board battery are physically integrated such that the system is operable as a stand-alone device.

The probe includes a structure that houses the ultrasound transduction elements and, optionally, associated electronic components. The probe comprises either a single or multiple transduction elements positioned in a physical cooperation with one another as to deliver ultrasound energy into the targeted root canal system. The configuration can be such that the ultrasound energy is delivered predominantly in one direction or, alternatively, such that ultrasound energy is delivered from several directions at once. The direction of energy delivery is defined from the outer surface of the tooth, the inner surface of the tooth, the biting surface of the tooth, or any combination thereof. (The “surfaces” of the tooth include those that are hidden from plain sight, i.e., the roots.) The physical configuration of transduction elements is either static or adjustable (e.g., to accommodate anatomical variations associated with intraoral environment). Mechanical and/or electronic user interfaces are integrated into the probe to allow for adjustments of the physical configuration of transduction elements and/or the electronic driving parameters of the device.

The transduction elements are designed to deliver energy to a specified targeted region. As such, active apertures of the elements are either geometrically focused or non-focused (for example, F-number ranging from about ½ and higher), each with effective surface area between about 3 mm² and about 300 cm². Transduction elements are composed of bulk lead-zirconate-titanate (PZT) piezoceramic or a PZT composite material and optionally poled to operate in the primary or higher-order thickness modes. Transduction elements can also be operated in primary or higher-order flexural modes. Transduction elements may be further sectioned (physically and/or electrically) to allow for phased array operation. The sonication frequency is chosen to be between about 100 kHz and about 4 MHz. The transduction elements may operate either at a single sonication frequency or at multiple sonication frequencies to elicit complex effects that may result in effective sonication frequencies outside of the aforementioned frequency range.

The probe incorporates a mechanism enabling coupling of the transduction elements to the patient. This either is accomplished using set-geometry compliant polymer standoff layers or with the use of patient-specific geometrically adapted polymer inserts. Coupling is established at the tooth surface, at the gingival surface, and/or at the outer surface of the face/cheek/mandible.

The electronic driving system (EDS) or circuitry includes a programmable actuator that has the capability to deliver low duty cycle (from about 0.01% to about 10%) actuation signals at relatively high instantaneous power levels (ranging, for example, from a few Watts to about 720 W) at the sonication frequency of interest. The EDS may be optionally enabled to switch between actuation parameters for effecting anesthesia or for disinfection. Depending on the power requirements, the EDS may include a benchtop component coupled electrically to the probe, or it may be integrated into the probe structure.

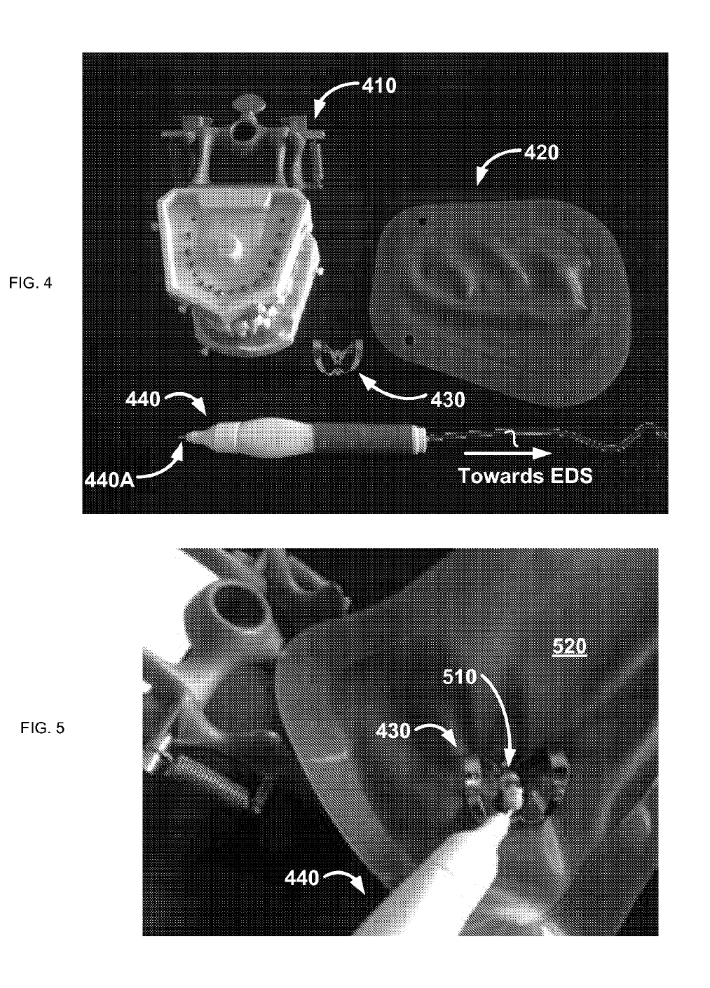

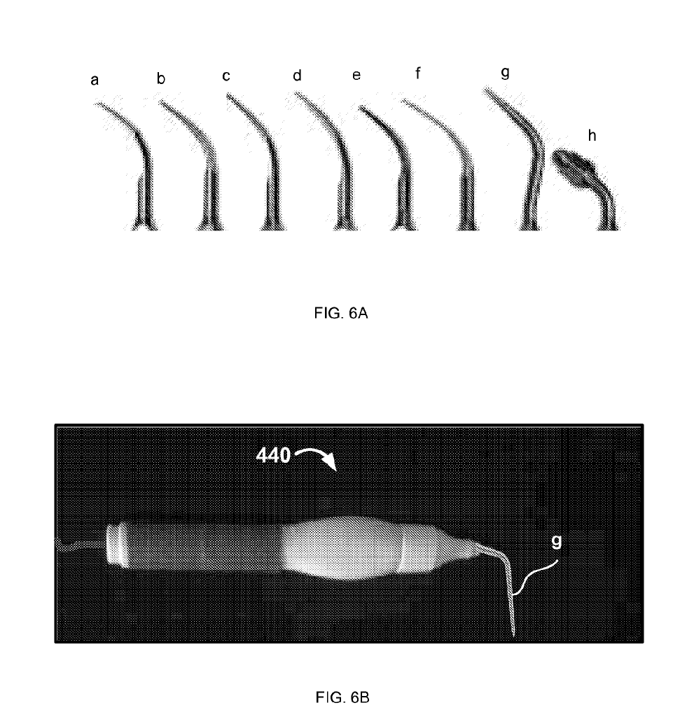

FIG. 4 provides an illustration to but one implementation of an apparatus for HIFU treatment. Shown are a dental model 410, a rubber dam 420, a retainer 430, and a HIFU probe 440 with coupling means 440A. FIG. 5 complements FIG. 4 by showing the application of the HIFU probe 440 to a tooth 510 during the endodontic treatment according to the idea of the invention, which entire treatment lasts for only one minute or less. FIGS. 6A and 6B show examples of interchangeable, replaceable, and differently-shaped tips (coupling means) structured for use with an embodiment of a HIFU device and the device itself, equipped with one of such tips.

Aggregately, FIGS. 4, 5, 6A, 6B illustrate the use of the device both in endodontics and with interchangeable elongated and curved tips for use in reaching to areas of pockets or loss of attachment during treatment of various stages of periodontal disease ADA/AAP Case types I-V, for treatment of peri-implantitis, and dental diseases that have bacterial etiology. Type I includes Gingivitis (associated with loss of attachment; bleeding on probing may be present); Type II includes Early Periodontitis (associated with pocket depth or attachment loss of about 3-4 mm; bleeding on probing may be present; localized area of gingival recession; possible grade I furcation involvement); Type III includes Moderate Periodontitis (characterized by pocket depths or attachment loss of about 4-6 mm; bleeding on probing; grade I or II furcation involvement; class I mobility); Type IV includes Advanced Periodontitis (characterized by pocket depths or attachment loss>6 mm; bleeding on probing; grade II or III furcation involvement; class II or III mobility); Type V includes Refractory & Juvenile Periodontitis (characterized by periodontitis not responding to conventional therapy or which recurs soon after treatment; juvenile forms of periodontitis).

A system governing the performance of the above-described method of the invention may include a processor controlled by instructions stored in a memory. The memory may be random access memory (RAM), read-only memory (ROM), flash memory or any other memory, or combination thereof, suitable for storing control software or other instructions and data. In addition, the invention may be partly embodied in software, the functions necessary to implement the invention may optionally or alternatively be embodied in part or in whole using firmware and/or hardware components, such as combinatorial logic, Application Specific Integrated Circuits (ASICs), Field-Programmable Gate Arrays (FPGAs) or other hardware or some combination of hardware, software and/or firmware components.

While the invention is described through the above-described exemplary embodiments, it will be understood by those of ordinary skill in the art that modifications to, and variations of, the illustrated embodiments may be made without departing from the inventive concepts disclosed herein. A methodology of applying the ultrasound for the purposes discussed above can be appropriately modified to optimize the efficiency of treatment (such as in the case of treating peri-implantitis). In a non-limiting specific example, the restoration crown can be removed from the dental implant and a replaceable attachment portion of the device can be placed directly on the metal portion of the dental implant (instead of the crown) to carry ultrasound through the metal to cause disinfection of bacteria and bacterial biofilms in the proximity of the metal root portion of the implant. Disclosed aspects, or portions of these aspects, may be combined in ways not listed above. Accordingly, the invention should not be viewed as being limited to the disclosed embodiment(s).

What is claimed is:

1. A method for operating a source of acoustic energy, the method comprising:
transmitting a focused acoustic beam generated by the source through a first tooth that is devoid of an access opening to an internal target area, the internal target area including a root-canal system of said first tooth, the source defining a confocal range of the beam that overlaps with the internal target area; and
maintaining said transmission through the first tooth for a period of time sufficient to disinfect endodontic tissue of the first tooth.

2. A method according to claim 1, wherein said maintaining includes not sonicating, with the focused beam, a second tooth that is immediately adjacent to the first tooth.

3. A method according to claim 1, further comprising transmitting the focused ultrasound beam through the first tooth to define ultrasound irradiance, in the internal target area that is aligned with a long axis of the first tooth's root and has dimensions of about 15 mm in length by about 5 mm in width.

4. A method according to claim 1, wherein the internal target area has a substantially cylindrical volume.

5. A method according to claim 1, wherein the source includes an array of ultrasound transducers.

6. A method according to claim 1, further comprising transmitting a substantially non-focused ultrasound beam generated by the source through a focusing means to produce the focused ultrasound beam.

7. A method according to claim 1, including wherein said maintaining includes maintaining said focused acoustic beam being transmitted through the first tooth for a period of time sufficient to treat at least one of (i) peri-implantitis in implant dentistry and (ii) ADA/AAP Case types I-V of periodontal disease.

8. A method for treatment of at least one of peri-implantitis in implant dentistry and ADA/AAP Case types I-V of periodontal disease, comprising a method according to claim 1.

9. A method for determining a parameter characterizing a tooth tissue, the method comprising:
sonicating a first tooth with a focused ultrasound beam incident on the first tooth externally such that an internal target area of the tooth is within a confocal range of the focused beam, the internal target area including a root-canal system of the tooth;
receiving, with a detector unit, a portion of the focused beam that has sonicated the first tooth to collect ultrasound data representing interaction of the focused beam with the tooth; and
determining, from the collected data, at least one of sound speed, mass density, and attenuation values associated with the tooth tissue.

10. A method according to claim 9, wherein the sonicating includes sonicating the internal target area through enamel and dentin, and wherein the first tooth is devoid of access opening through enamel and dentin to the internal target area.

11. A method according to claim 9, wherein the sonicating includes sonicating a first tooth such that a second tooth that is immediately adjacent to the first tooth is not sonicated.

12. A method for treatment of at least one of peri-implantitis in implant dentistry and ADA/AAP Case types I-V of periodontal disease, comprising a method according to claim 9.

13. An article of manufacture comprising:
a source of ultrasound including means defining, in operation, a focused ultrasound beam generated by the source; and
a non-transitory tangible computer-readable medium having computer-readable program code thereon, the computer-readable program code including
program code for acquiring data that represent the focused ultrasound beam that has been transmitted through a tooth, the tooth being devoid of access opening to a root-canal system of the tooth; and
program code for determining a parameter characterizing a process of sonication of the tooth with the focused ultrasound beam in such a manner as to disinfect the root-canal system.

14. An article of manufacture according to claim 13, wherein said source includes a probe having a proximal end, a distal end, and an elongated curved replaceable attachment dimensioned to fit in an area of disattachment between gum and the tooth subject to ADA/AAP Case types I-V of periodontal disease and replaceably affixed to the distal end such as to transmit said focused beam towards said tooth, the proximal end being operably extended to an electronic driving system

15. An article of manufacture according to claim 14, wherein the electronic driving system includes a programmable actuator operable to deliver , to said attachment, actuation signals with duty cycle variable from about 0.01% to about 10% at an instantaneous power level ranging from about one Watt to about 720 W at a predetermined sonication frequency.

16. An article of manufacture according to claim 13, wherein the computer-readable program code further includes program code for determining a parameter characterizing a process of sonication of the tooth with the focused ultrasound beam in such a manner as to determine ultrasound parameters sufficient to cause at least one of local anesthesia and nerve blocking of tissue associated with the tooth.

17. An article of manufacture according to claim 13, wherein the computer-readable program code further includes program code for determining a parameter characterizing nerve blocking effectuated by sonication of a tooth with the focused ultrasound beam.

18. An article of manufacture according to claim 13, wherein the computer-readable program code further includes program code for governing an operation of the source of ultrasound.

19. A method for altering sensitivity of oral tissue, the method comprising:
sonicating a region of interest (ROI) of the tissue with a focused ultrasound beam incident on the tissue externally such that the ROI remains within a confocal range of the focused beam;
receiving, with a detector unit, a portion of the focused beam that has sonicated the ROI to collect ultrasound data representing interaction of the focused beam with the tissue; and
determining, from the collected data, at least one parameter describing at least one parameter describing ultrasound such that sonicating of the tissue with an ultrasound beam characterized by the at least one parameter is sufficient to cause at least one of local anesthesia and nerve blocking of at least the ROI.

20. A method according to claim 19, wherein the oral tissue includes a first tooth.

21. A method according to claim 20, wherein the ROI includes a root-canal system of the tooth.
